(19) United States
(12) Patent Application Publication
(10) Pub. No.: US 2025/0107775 A1
(43) Pub. Date: Apr. 3, 2025

(54) MIXED REALITY-BASED ULTRASONIC IMAGE DISPLAY DEVICE, METHOD, AND SYSTEM

(71) Applicant: KOREA UNIVERSITY RESEARCH AND BUSINESS FOUNDATION, Seoul (KR)

(72) Inventors: Sung-Joon PARK, Ansan-si, Gyeonggi-do (KR); In-Tae HWANG, Gwangmyeong-si, Gyeonggi-do (KR)

(21) Appl. No.: 18/703,422

(22) PCT Filed: Feb. 10, 2023
(86) PCT No.: PCT/KR2023/002000

§ 371 Date: Apr. 22, 2024

(30) Foreign Application Priority Data
Feb. 16, 2022 (KR) 10-2022-0020022

(51) Int. Cl. A61B 8/00; A61B 90/00; A61B 90/50; G06T 7/13; G06T 7/90; G06T 19/00; G06T 19/20

(52) U.S. Cl. CPC A61B 8/463; A61B 8/4254; G06T 7/13; G06T 7/90; G06T 19/006; G06T 19/20; A61B 2090/365; A61B 2090/367; A61B 2090/502; G06T 2200/24; G06T 2207/10024; G06T 2207/10136; G06T 2207/30004; G06T 2210/41; G06T 2210/62; G06T 2219/2004

(57) ABSTRACT

An apparatus, method, and system for displaying an ultrasound image based on mixed reality are described. The method of displaying an ultrasound image based on mixed reality according to an exemplary embodiment includes obtaining the ultrasound image captured through an ultrasound device; obtaining localization information of a probe of the ultrasound device; converting the ultrasound image into a composite image to be output based on the mixed reality; and outputting the composite image by overlaying the composite image on a reference image captured to include the probe.

### TECHNICAL FIELD

The present invention relates to an apparatus, method, and system for displaying an ultrasound image based on mixed reality. For example, the present invention relates to a system for increasing intuitive examination and examination efficiency by playing results of existing ultrasound images to match a subject's affected area using mixed reality equipment and by linking the ultrasound images with a localization of a transducer to improve intuitive understanding of lesions without secondary interpretation of anatomy.

### BACKGROUND ART

An ultrasound system is a system that displays an inside of a human body in real time through images and is used to determine whether there are any abnormalities in organs at an early stage. Currently, the global market size for ultrasound diagnostic devices is expected to grow to around 7 trillion won by 2020, and the domestic general-purpose ultrasound diagnostic device market is estimated at around 470.6 billion won as of 2019.

Ultrasound imaging magnifies and images the degree of reflection of sound waves sent through the skin, so an operator only sees the outside of the body, and a shape projected through the image is a cross-sectional view of a tissue within a certain depth of the skin. Accordingly, an operation of overlapping a scene looking down and the cross-sectional view of the image in real time should be continuously performed. In general, the affected area and the ultrasound image display are not located within the operator's line of sight, so it is difficult for the operator to constantly turn his/her head to look at the two areas alternately.

Because of these technical characteristics, the results of the ultrasound capturing appear differently depending on the technical skill of the operator, which is a field that requires years of training. The main difficulties in training are due to a discrepancy between an act of an operator bringing an ultrasound probe into contact with an affected area to find and capture abnormal areas and an image from a screen that plays the captured image.

That is, in the ultrasound procedure, the main learning curve shows that the level of difficulty is the highest level at which real-time understanding of the anatomy and scope of ultrasound images linked to a localization of a transducer is possible. In other words, spatial cognitive ability is required to imagine the localization of the ultrasound image in an operator's head and understand the anatomical structure, which is also a very difficult barrier for beginners.

As a way to solve the above-described difficulties, a technique of matching positions by directly projecting result values of ultrasound images onto affected areas may be considered. The technique involves directly projecting images obtained from ultrasound diagnosis onto the affected areas. The technique has the limitation that, due to the nature of the ultrasound diagnosis which is performed while lying on a bed, the image has no choice but to be projected downward from a ceiling, and has the disadvantage that, due to the nature of image projection using a projector, it is nothing more than a monitor of an ultrasound machine, and therefore, an operator has no choice but to confirm an image rotated 90° regardless of a direction of an ultrasound probe.

The background technology of the present invention is disclosed in Korean Patent No. 10-1231926.

### DISCLOSURE

### Technical Problem

The present invention provides an apparatus, method, and system for displaying an ultrasound image based on mixed reality that enables intuitive ultrasound examination and ultrasound-guided procedures by linking the ultrasound image to a localization of a probe (transducer) of an ultrasound device to express the ultrasound image in a field of view of the mixed reality, and at the same time, accumulate and display the ultrasound image in three dimensions.

However, the technical problems to be achieved by the exemplary embodiments of the present invention are not limited to the technical problems as described above, and other technical problems may exist.

### Technical Solution

According to an aspect of the present invention, a method of displaying an ultrasound image based on mixed reality may include: obtaining the ultrasound image captured through an ultrasound device; obtaining localization information of a probe of the ultrasound device; converting the ultrasound image into a composite image to be output based on the mixed reality; and outputting the composite image by overlaying the composite image on a reference image captured to include the probe.

In the outputting, the composite image may be overlaid to be adjacent to an end area of the probe that appears in the reference image.

In the outputting, the composite image may be overlaid on the reference image displayed on a screen of a head-mounted display worn by a user.

According to an exemplary embodiment of the present invention, the method of displaying an ultrasound image based on mixed reality may include: obtaining position information and field of view information of the head-mounted display; and correcting the overlaid position of the composite image based on the position information and the field of view information.

According to an exemplary embodiment of the present invention, the method of displaying an ultrasound image based on mixed reality may include: receiving user input for capturing the composite image; and displaying a fixed image in which the composite image is continuously maintained at a predetermined fixed position on the reference image based on the user input.

In the displaying of the fixed image, the fixed image may be output by applying a predetermined transparency to the composite image.

According to an exemplary embodiment of the present invention, the method of displaying an ultrasound image based on mixed reality may include: extracting three-dimensional (3D) boundary information on the captured object of the ultrasound device based on color information of each of the plurality of fixed images in a state where the fixed image is individually displayed at a plurality of different fixed positions based on the user input applied multiple times; and updating the composite image to include a virtual 3D image corresponding to the captured object based on the 3D boundary information.

According to an exemplary embodiment of the present invention, the method of displaying an ultrasound image based on mixed reality may further include: receiving the user input for adjusting the position of the composite image; and adjusting the overlaid position of the composite image on the reference image based on the user input.

According to another aspect of the present invention, a method of displaying an ultrasound image based on mixed reality may include: obtaining the ultrasound image captured through an ultrasound device; obtaining localization information of a probe of the ultrasound device; converting the ultrasound image into a composite image to be output based on the mixed reality in consideration of the localization information; and transmitting the composite image to a user terminal.

The user terminal may include a head-mounted display worn by a user operating the ultrasound device.

According to an exemplary embodiment of the present invention, the method of displaying an ultrasound image based on mixed reality may include: receiving user input for capturing the composite image; and transmitting, to the user terminal, a fixed image in which the composite image is continuously maintained at a predetermined fixed position on the reference image based on the user input.

According to an exemplary embodiment of the present invention, the method of displaying an ultrasound image based on mixed reality may include: extracting three-dimensional boundary information on the captured object of the ultrasound device based on color information of each of the plurality of fixed images in a state where the fixed image is individually displayed at a plurality of different fixed positions based on the user input applied multiple times; and transmitting, to the user terminal, the composite image updated to include a virtual 3D image corresponding to the captured object based on the 3D boundary information.

According to still another aspect of the present invention, an apparatus for providing ultrasound image display service based on mixed reality may include: a receiving unit that obtains an ultrasound image captured through an ultrasound device and obtains localization information of a probe of the ultrasound device; a processing unit that converts the ultrasound image into a composite image to be output based on the mixed reality in consideration of the localization information; and a transmitting unit that transmits the composite image to a user terminal.

According to still yet another aspect of the present invention, a system for displaying an ultrasound image based on mixed reality may include: an ultrasound device that captures an ultrasound image using a probe; a service providing device that obtains the ultrasound image and localization information of the probe, and converts the ultrasound image into a composite image to be output based on the mixed reality; and a user terminal that receives the composite image from the service providing device and outputs the composite image by overlaying the composite image on a reference image captured to include the probe.

According to an exemplary embodiment of the present invention, the system for displaying an ultrasound image based on mixed reality may include: a tracking device that is disposed relative to the probe, measures the localization information, and transmits the measured localization information to the service providing device.

The means for solving the problem described above are merely exemplary and should not be construed as limiting the present invention. In addition to the exemplary embodiments described above, additional exemplary embodiments may exist in the drawings and detailed description of the disclosure.

### Advantageous Effects

According to an apparatus, method, and system for displaying an ultrasound image based on mixed reality of the present invention described above, by linking the ultrasound image to a localization of a probe (transducer) of an ultrasound device to express the ultrasound image in a field of view of the mixed reality, and at the same time, accumulate and display the ultrasound image in three dimensions, it is possible to perform intuitive ultrasound examination and ultrasound-guided procedures.

According to the present invention described above, it is possible to intuitively and interactively express real-time images to the user by expressing an image in real time so that the image matches a position of the probe (transducer) of the ultrasound device, generate the images as a three-dimensional object having a volume and display the image three-dimensionally by processing the images into a transparent image and accumulating the image in real time, and assist in intuitively identifying a target to be approached for a procedure by allowing a user to three-dimensionally understand a structure (e.g., blood vessels, muscles, fascia, etc.) of an object to be captured reflected in the ultrasound image.

According to the present invention described above, it is possible to increase understanding of structural features of the captured object and observe specific lesions more closely based on a virtual ultrasound image that matches a position of a probe and an affected area by allowing an operator performing ultrasound examination or ultrasound-guided procedures to recognize that the ultrasound image is output from an end portion of the probe of the ultrasound device.

However, the effects obtainable herein are not limited to the effects described above, and other effects may exist.

### DESCRIPTION OF DRAWINGS

FIG. 1 is a schematic configuration diagram of a system for displaying an ultrasound image based on mixed reality according to an exemplary embodiment of the present invention.

FIG. 2 is an exemplary diagram of a mixed reality-based screen in which a composite image is overlaid on a reference image and output through a user terminal.

FIG. 3 is a conceptual diagram for describing a composite image including a virtual three-dimensional (3D) image corresponding to a captured object.

FIG. 4 is an exemplary diagram of a fixing member for combining a probe and a tracking device.

FIG. 5 is a schematic configuration diagram of an apparatus for providing ultrasound image display service based on mixed reality according to an exemplary embodiment of the present invention.

FIG. 6 is an operation flowchart of a method of displaying an ultrasound image based on mixed reality according to an exemplary embodiment of the present invention.

FIG. 7 is an exemplary diagram of an image in which the composite image is overlaid on the reference image.

FIG. 8 is a detailed operation flowchart of a process in which the system for displaying an ultrasound image based on mixed reality according to the exemplary embodiment of the present invention displays a fixed image and forms a virtual three-dimensional image of a captured object based on a plurality of fixed images.

### BEST MODE

Hereinafter, exemplary embodiments of the present invention will be described in detail with reference to the accompanying drawings so that those skilled in the art to which the present invention pertains may easily practice. However, the present invention may be implemented in various different forms, and is not limited to the exemplary embodiments described herein. In addition, in the drawings, portions unrelated to the description will be omitted to clearly describe the present invention, and similar portions will be denoted by similar reference numerals throughout the specification.

In addition, throughout the present specification, when any one part is referred to as being “connected to” another part, it means that any one part and another part are “directly connected to” each other or are “electrically connected to” or “indirectly connected to” each other with the other part interposed therebetween.

Throughout the present specification, when any member is referred to as being positioned “on”, “at upper portion”, “at upper end”, “below”, “at lower portion”, “at lower end” of another member, it includes not only a case in which any member and another member are in contact with each other, but also a case in which the other member is interposed between any member and another member.

Through the present specification, unless explicitly described otherwise, “comprising” any components will be understood to imply the inclusion of other components rather than the exclusion of any other components.

The present invention relates to an apparatus, method, and system for displaying an ultrasound image based on mixed reality. For example, the present invention relates to a system for increasing intuitive examination and examination efficiency by playing results of existing ultrasound images to match a subject's affected area using mixed reality equipment and by linking the ultrasound images with a localization of a probe to improve intuitive understanding of lesions without secondary interpretation of anatomy.

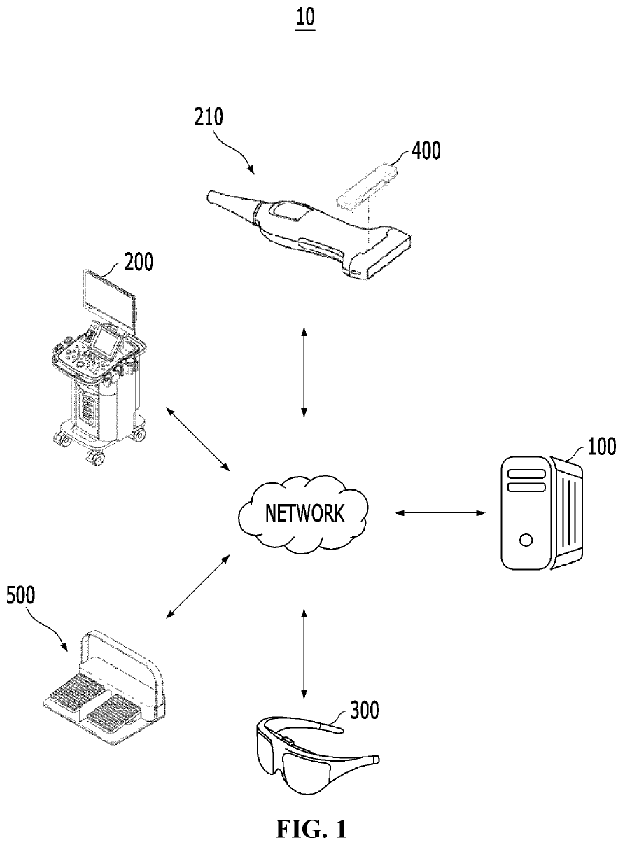

FIG. 1 is a schematic configuration diagram of a system for displaying an ultrasound image based on mixed reality according to an exemplary embodiment of the present invention.

Referring to FIG. 1, a system 10 (hereinafter referred to as ‘display system 10’) for displaying an ultrasound image based on mixed reality according to an exemplary embodiment of the present invention may include an apparatus 100 (hereinafter referred to as ‘service providing device 100’) for providing ultrasound image display service based on mixed reality according to an exemplary embodiment of the present invention, an ultrasound device 200, a user terminal 300, a tracking device 400, and an input device 500.

The service providing device 100, the ultrasound device 200, the user terminal 300, the tracking device 400, and the input device 500 may communicate with each other through a network 20. The network 20 refers to a connection structure capable of exchanging information between each node, such as terminals and servers. Examples of such a network 20 include a 3rd generation partnership project (3GPP) network, a long term evolution (LTE) network, a 5G network, a world interoperability for microwave access (WIMAX) network, Internet, a local area network (LAN), a wireless local area network (wireless LAN), a wide area network (WAN), a personal area network (PAN), a Wi-Fi network, a Bluetooth network, a satellite broadcasting network, an analog broadcasting network, a digital multimedia broadcasting (DMB) network, and the like, but are not limited thereto.

In the description of the exemplary embodiment of the present invention, the ultrasound device 200 may refer to equipment that shoots sound waves and interprets reflected signals to obtain information on organs located under a skin of a capturing target (e.g., patient, etc.) and displays a cross-sectional image. In addition, the ultrasonic device 200 includes a probe 210 that is provided in a size that may be held in a user's (operator's) hand and has a sensor portion provided at a lower end thereof. Due to the nature of this device, the characteristics of the ultrasound image obtained vary depending on an angle at which the probe 210 is in contact with a body to be captured and strength with which probe 210 is pressed. Accordingly, the user should perform an operation of accurately determining anatomical characteristics of a human body and conditions of a patient and obtaining images by applying an appropriate force to the affected area, and should manipulate equipment in various manners to instantly recognize an area that is determined to be abnormal and obtain more detailed images. Therefore, many years of various training and experience are required to accurately find a lesion, and the accuracy of examination varies depending on the operator's skill level.

Furthermore, in the case of the conventional ultrasound device, there is a limitation in that the equipment displaying (playing) the obtained ultrasound images is disposed in a separate external position regardless of the probe 210 manipulated by the operator. That is, in the case of the conventional ultrasound device, the operator should perform an operation of moving the device to the affected area using his or her hand while watching the obtained image in real time, and tracking the exact position by looking at the image and the affected area alternately. Accordingly, prior anatomical knowledge and the tactile sensation of the hand touching the affected area also become important.

The user terminal 300 may be, for example, a smartphone, a smartpad, a tablet PC, etc., and all types of wireless communication devices such as personal communication system (PCS), global system for mobile communication (GSM), personal digital cellular (PDC), personal handyphone system (PHS), personal digital assistant (PDA), international mobile telecommunication (IMT)-2000, code division multiple access (CDMA)-2000, W-code division multiple access (W-CDMA), and wireless broadband Internet (Wibro) terminals.

In particular, according to an exemplary embodiment of the present invention, the user terminal 300 may be an AR/VR device capable of playing the mixed reality-based ultrasound image of the present invention. Specifically, the AR/VR device may refer to a head-mounted display terminal. In this regard, the user terminal 300 may output a composite image based on the ultrasound image received from the service providing device 100 by overlaying the composite image on a reference image to be described later.

In addition, in the description of the exemplary embodiment of the present invention, the tracking device 400 may be a device that is disposed with respect to the probe 210 of the ultrasound device 200, measures localization information of the probe 210, and transmits the measured localization information to the service providing device 100. More specifically, the localization information measured by the tracking device 400 may include position information, angle (tilt) information, movement speed information, path information, etc., of the probe 210 in a three-dimensional space.

That is, the tracking device 400, which includes an optical camera and inertial specific equipment, may be attached to the probe 210 to track the position of the probe 210 of the ultrasonic device 200 in real time, and may be disposed so that the localization information including movement measurement values, etc., of the tracking device 400 may be interpreted as localization information according to the movement of the probe 210 by matching translational and rotational directions of the probe 210 and the tracking device 400 after the attachment.

Meanwhile, in the description of the exemplary embodiment of the present invention, the tracking device 400 is a separate device from the probe 210 of the ultrasound device 200, and is disposed (for example, it is coupled to the probe 210 using a predetermined fixing member (e.g., screw-hole coupling structure, etc.) and has a form that is coupled to the probe 210) with respect to the probe 210 to allow the service providing device 100 to receive the localization information measured with respect to the tracking device 400 and obtains the localization information of the probe 210 of the ultrasonic device 200 using the received localization information, but is not limited thereto.

As another example, according to the implementation example of the present invention, the tracking device 400 may be referred to as a sensor module such as an inertial sensor that is built into (mounted on) the probe 210 and measures the localization information of the probe 210 and transmits the measured localization information to the service providing device 100.

In addition, in the description of the exemplary embodiment of the present invention, the input device 500 may be a device that is provided to convert the ultrasound image obtained from the ultrasound device 200 with respect to the service providing device 100 into a two-dimensional (2D) or three-dimensional (3D) composite image (composite ultrasound image) tailored to the localization information of the probe 210 and apply a predetermined user input for setting and applying details applied during playing the composite image through the user terminal 300.

As an example, according to an exemplary embodiment of the present invention, the input device 500 may be a device that is provided in the form of a pedal (foot pedal) as illustrated in FIG. 1 to determine whether the user input is applied based on the pressure that a user (operator) applies to the pedal, but is not limited thereto. As another example, the input device 500 may include a control panel including a button or may be provided in various types to receive a wide range of user inputs such as identifying a user's (operator's) arm, hand, face, etc., determining gestures, or determining the user inputs based on the user's (operator's) voice. For reference, the input device 500 may be referred to differently as an ‘interaction device’ or the like. Hereinafter, specific functions and operations of the display system 10 will be described in detail.

The service providing device 100 may obtain the ultrasound image captured through the ultrasound device 200. For example, the service providing device 100 may receive a real-time ultrasound image captured using the probe 210 from the ultrasound device 200.

In addition, the service providing device 100 may obtain the localization information of the probe 210 of the ultrasound device 200. According to an exemplary embodiment of the present invention, the service providing device 100 may receive the localization information of the probe 210 from the tracking device 400 disposed with respect to the probe 210 or receive the localization information of the tracking device 400 which may be replaced by the localization information of the probe 210.

In addition, the service providing device 100 may convert the obtained ultrasound image into the composite image (composite ultrasound image) for output based on the mixed reality (MR) through the user terminal 300.

For reference, in the description of the exemplary embodiments of the present invention, the ‘mixed reality’ is a term that includes the meaning of augmented reality (AR) which adds virtual information based on reality and augmented virtuality (AV) which adds real information to a virtual environment, and refers to a technology that combines the reality and virtuality to create a new environment where real and virtual objects coexist and allows users to experience various types of digital information more realistically by interacting with the environment in real time. In this regard, the display system 10 disclosed herein is designed to overcome the problems of the conventional ultrasound device 200 that adds (combines) a composite ultrasound image (‘composite image’) converted from a captured ultrasound image using the probe 210 to an image (‘reference image’) of the probe 210 that exists in the actual space where ultrasound examination or ultrasound-guided treatment is performed to allow an operator (user) to recognize that the ultrasound image is displayed adjacent to an end portion (e.g., lower end portion) of the probe 210.

In addition, the service providing device 100 may transmit the converted composite image to the user terminal 300 and output the composite image by overlaying the composite image on the reference image captured by the user terminal 300 to include the probe 210.

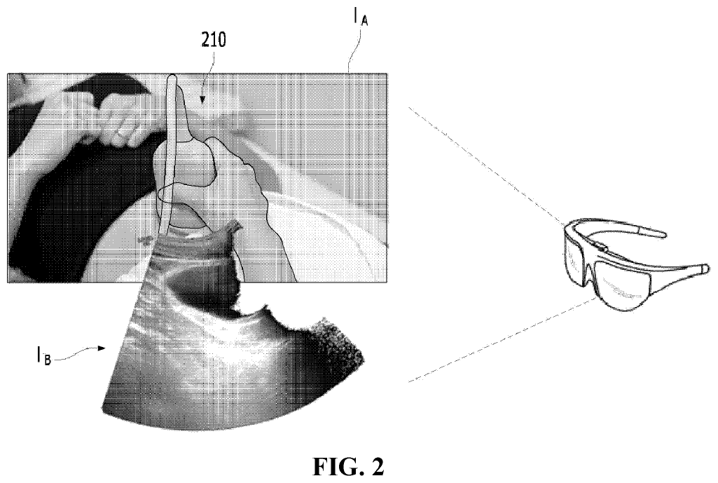

In this regard, FIG. 2 is an exemplary diagram of a mixed reality-based screen in which the composite image is overlaid on the reference image and output through the user terminal.

Referring to FIG. 2, the service providing device 100 may determine a size, a position, and a direction where overlaying a composite image I_{B} on a reference image I_{A} by considering the localization information of the probe 210 so that the composite image (virtual image, I_{B}) in which an original ultrasound image is converted into a virtual ultrasound image based on the localization information of the probe 210, the position information and field of view information of the head-mounted display 300 worn by the user, etc., may be displayed by overlaying the reference image (real image, I_{A}) captured to include the probe on the screen of the user terminal 300, in particular, the head-mounted display (HMD) 300 worn by the user.

Specifically, according to an exemplary embodiment of the present invention, the service providing device 100 may calculate the display size, position, and direction of the composite image based on the localization information of the probe 210 so that the composite image may overlay adjacent to an end area of the probe that appears in the reference image.

Here, the fact that the composite image is adjacent to the end area of the probe refers to that, as illustrated in FIG. 2, the composite image is arranged to extend downward from a lower end surface of the probe 210 that contacts the skin of the capturing target (patient, etc.) of the probe 210 based on an extension direction of the probe 210.

In addition, the service providing device 100 may determine the output (overlaying) size, position, and direction of the composite image so that the composite image is output in a direction perpendicular to the contact surface by considering an extension direction of a contact surface where the probe 210 and the affected area of the capturing target are in contact with each other based on the localization information of the probe 210. Accordingly, the user may intuitively determine how a scanning direction of the ultrasound image actually changes depending on the degree to which the user manipulates the probe 210 (e.g., tilts the probe 210, changes the position of the probe 210, etc.) by displaying the composite image perpendicular to the affected area to correspond to the scanning direction of the obtained ultrasound image using the probe 210.

In addition, according to an exemplary embodiment of the present invention, the service providing device 100 may obtain the position information and field of view information of the head-mounted display (user terminal, 300), and correct the position where the composite image is overlaid on the reference image based on the obtained position information of the HMD and the user's (operator's) field of view information.

In addition, according to an exemplary embodiment of the present invention, the service providing device 100 may be operated to receive the user input for adjusting the position of the composite image from at least one of the user terminal 300 and the input device 500, and adjust the overlaid position of the composite image on the reference image based on the received user input for position adjustment.

In other words, when there is a gap between the actual position of the probe 210 which can be confirmed on the display screen where the reference image and the composite image are overlaid and the position where the composite image is displayed, the service providing device 100 may receive the user input for correcting the output (overlaying) position of the composite image through the input device 500 or the user terminal 300.

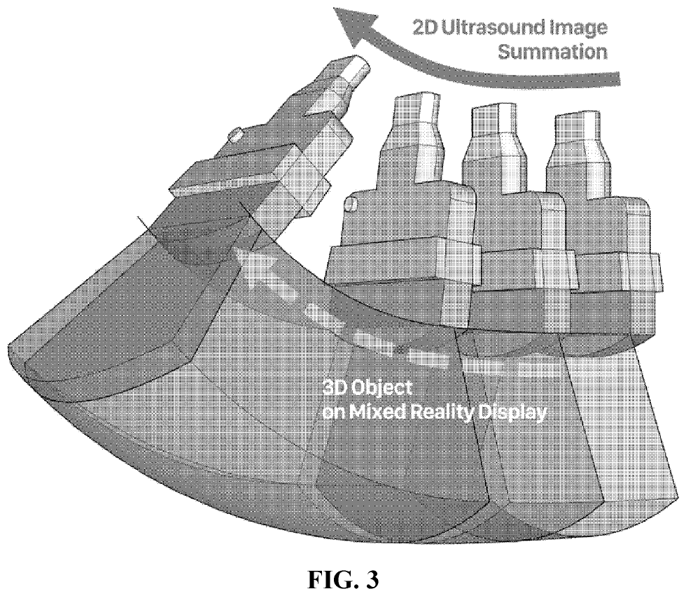

FIG. 3 is a conceptual diagram for describing the composite image including the virtual 3D image corresponding to the captured object.

Referring to FIG. 3, the service provision device 100 may continuously maintain and display a composite image converted from an ultrasound image captured at a specific time/position at a predetermined fixed position on the reference image based on the captured input (specifically, the user input for capturing the composite image overlaid on the reference image) applied to at least one of the user terminal 300 and the input device 500, and display a composite image containing a virtual 3D image corresponding to the captured object through the user terminal 300 based on multiple composite images or composite image sequences obtained continuously in time series.

Specifically, the service providing device 100 may receive the user input for capturing the composite image to display a fixed image in which the composite image is continuously maintained at a predetermined fixed position on the reference image based on the user input, and output the fixed image with a predetermined transparency applied to the composite image so as not to obstruct the operator's (user's) view of field who looks at the affected area of the capturing target (patient, etc.).

In addition, the service providing device 100 may extract 3D boundary information on the captured object of the ultrasound device based on color information of each of the plurality of fixed images in the state where the fixed image is individually displayed at a plurality of different fixed positions based on the user input (captured input) applied multiple times.

In addition, the service providing device 100 may update the composite image to include a virtual 3D image corresponding to the captured object based on the extracted 3D boundary information.

That is, the service providing device 100 may record a played position of the composite image converted from the ultrasound image by the signal transmitted through the interaction device (input device, 500) such as the button, gesture, or pedal, process images stored at each position into images with transparency and display the processed images as fixed images when input values are continuously transferred several times or more or for several hours or more, compare color information, such as gray scale, of each pixel that constitutes the fixed images to extract points corresponding to differences above a certain level and then extract outlines of the captured objects (e.g., blood vessels, muscles, fascia, organs, etc.) in a manner to connect the extracted points, accumulate the outlines in real time to be generated as a volume object, and update the generated volume object to the composite image that may be confirmed in three dimensions and output the composite image, thereby assisting the user to three-dimensionally determine the structures (vascular, muscle, fascia, etc.) on the ultrasound image.

In addition, the service providing device 100 may provide a function of adjusting the degree of gray scale, which is the standard for extraction, through control through the interaction. More specifically, the service providing device 100 may variably adjust an extraction reference value (e.g., gray scale value, etc.) for extracting the plurality of fixed images based on the user's adjustment input (e.g., gesture-based slide adjustment, etc.) applied through the interaction device 500.

That is, the display system 10 disclosed herein may include a function of storing the position of the affected area through the interaction using the input device 500, such as the pedal, so that the ultrasound image is fixed to the spatial position, and accumulating images of the plurality of consecutive ultrasound images and converting the accumulated images into a 3D model.

Meanwhile, in the above description, the exemplary embodiment in which the display device 10 augments (overlays) the ultrasound image and displays the augmented ultrasound image on the screen of the head-mounted display (HMD) worn by the user has been mainly described, but when the display system 10 disclosed herein is the ultrasonic device 200 of a type that may be output to an external display device through a separate cable, etc., in addition to a display module responsible for an image output function of a main body of the ultrasonic device 200 according to the implementation example of the present invention, the display system 10 may include a playback device (not illustrated) that may receive the obtained ultrasound image as a result of examination and play and record the received ultrasound image. In this regard, the display system 10 may include a conversion module that converts an image signal transmitted through an image conversion cable connecting an image output terminal of the ultrasound device 200 and the playback device so that the image signal may be viewed on the playback device, and software that plays and records the resulting images.

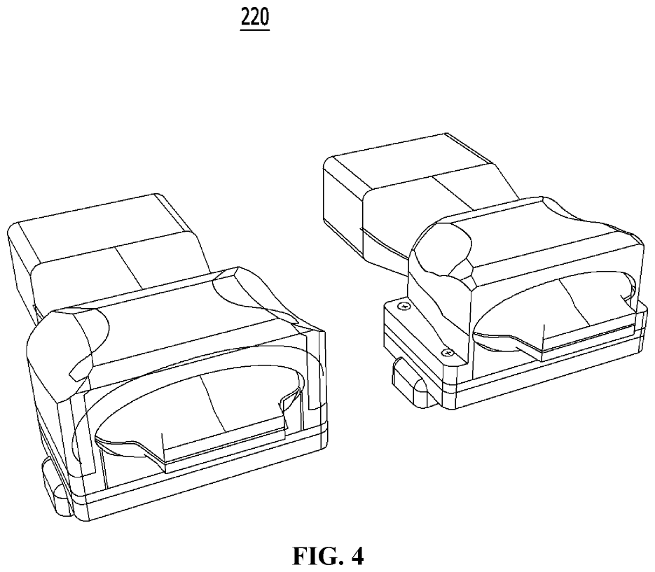

FIG. 4 is an exemplary diagram of a fixing member for combining a probe and a tracking device.

Referring to FIG. 4, a fixing member 220 for combining the probe 210 and the tracking device 400 may have a hole with the same width as a screw hole pre-drilled in the probe 210 so that it may be fixed to the equipment that tracks the movement of the probe without any clearance or gap in movement, without interfering with the act of holding the probe 210 with a transducer's hand, and may be fastened by tightening a bolt that fits the screw hole, but is not limited thereto.

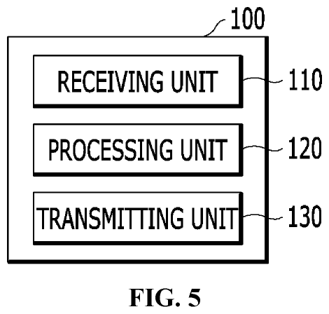

FIG. 5 is a schematic configuration diagram of an apparatus for providing ultrasound image display service based on mixed reality according to an exemplary embodiment of the present invention.

Referring to FIG. 5, the service providing device 100 may include a receiving unit 110, a processing unit 120, and a transmitting unit 130.

The receiving unit 110 may obtain the ultrasound image captured through the ultrasound device 200. In addition, the receiving unit 110 may obtain the localization information of the probe 210 of the ultrasound device 200.

The processing unit 120 may convert the ultrasound image into a composite image for output based on mixed reality by considering the obtained localization information of the probe 210.

The transmitting unit 130 may transmit the converted composite image to the user terminal 300.

Hereinafter, the operation flow of the present invention will be briefly described based on the details described above.

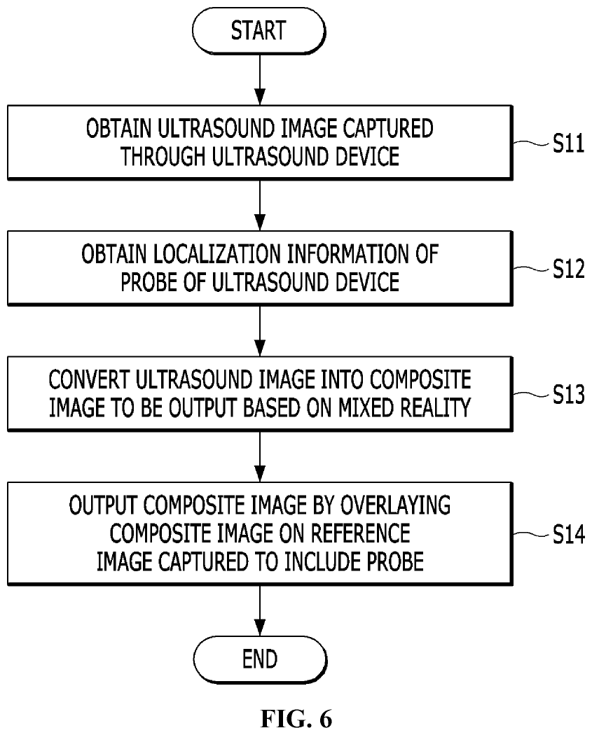

FIG. 6 is an operation flowchart of a method of displaying an ultrasound image based on mixed reality according to an exemplary embodiment of the present invention.

The method of displaying an ultrasound image based on mixed reality illustrated in FIG. 6 may be performed by the display system 10 described above. Therefore, even if the contents are omitted below, the contents described with respect to the display system 10 may also be equally applied to the description of the method of displaying an ultrasound image based on mixed reality.

Referring to FIG. 6, in step S11, the service providing device 100 may obtain the ultrasound image captured through the ultrasound device 200.

Next, in step S12, the service providing device 100 may obtain the localization information of the probe 210 of the ultrasound device 200.

Specifically, in step S12, the service providing device 100 may receive the localization information of the probe 210 including the position information, rotation information, and movement speed information, etc., of the probe 210 from the tracking device 400 disposed with respect to the probe 210.

Next, in step S13, the service providing device 100 may convert the ultrasound image obtained in step S11 into the composite image for output based on mixed reality.

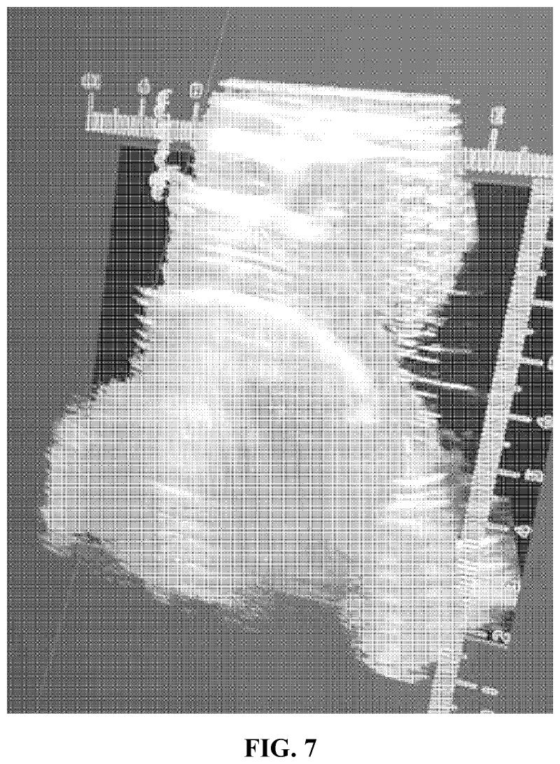

Meanwhile, FIG. 7 is an exemplary diagram of the image in which the composite image is overlaid on the reference image.

Next, in step S14, referring to FIG. 7, the user terminal 300 may output the composite image generated in step S13 by overlaying the composite image with the reference image captured to include the probe 210.

Specifically, in step S14, the service providing device 100 may transmit the composite image converted from the ultrasound image to the user terminal 300 and output the composite image and reference image in the mixed reality form through the display module of the user terminal 300, and in step S14, the user terminal 300 may overlay the composite image so that the composite image is adjacent to the end area of the probe 210 that appears in the reference image.

In the above description, steps S11 to S14 may be further divided into additional steps or combined into fewer steps, according to the implementation example of the present invention. Also, some steps may be omitted if necessary, and an order between operations may be changed.

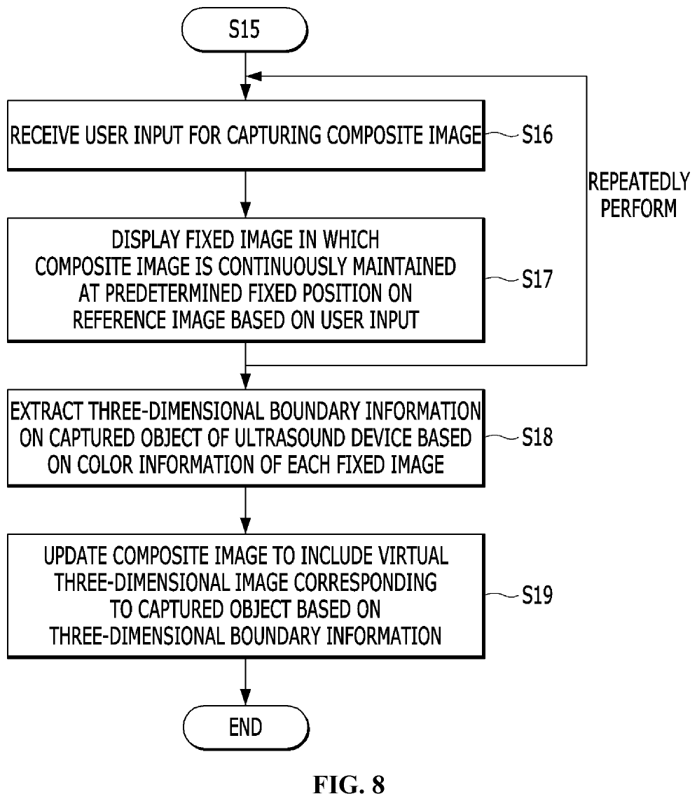

FIG. 8 is a detailed operation flowchart of a process in which the system for displaying an ultrasound image based on mixed reality according to the exemplary embodiment of the present invention displays a fixed image and forms a virtual three-dimensional image of a captured object based on a plurality of fixed images.

The process of displaying the fixed image illustrated in FIG. 8 and forming the virtual three-dimensional image of the captured object may be performed by the display system 10 described above. Therefore, even if the contents are omitted below, the contents described for the display system 10 may be equally applied to the description of FIG. 6.

Referring to FIG. 8, in step S16, the service providing device 100 may receive the user input for capturing the composite image. As an example, the service providing device 100 may receive the user input, which is input to the input device 500, from the input device 500.

Next, in step S17, the service providing device 100 may generate a fixed image that allows the composite image to be continuously maintained at a predetermined fixed position (e.g., the position where the composite image is displayed at the time the user input is applied, etc.) on the reference image based on the user input obtained in step S11, transmit the generated fixed image to the user terminal 300, and display the fixed image.

In addition, according to an exemplary embodiment of the present invention, in step S17, the user terminal 300 may output the fixed image by applying a predetermined transparency to the composite image.

Meanwhile, the above-described steps S16 and S17 may be repeatedly performed multiple times based on the user input to capture the composite images that are each applied when the probe 210 probes different positions during the ultrasound examination, etc., or performed continuously and repeatedly for a certain period of time when the user input is a type for continuously capturing the composite images (‘repeated performance’ in FIG. 6).

Next, in step S18, the service providing device 100 may extract the 3D boundary information on the captured object of the ultrasound device 200 based on the color information of each of the plurality of fixed images in the state where the fixed image is individually displayed at the plurality of different fixed positions based on the user input applied multiple times.

Next, in step S19, the service providing device 100 may transmit to the user terminal 300 the updated composite image to include the virtual 3D image corresponding to the captured object based on the 3D boundary information derived through step S18.

In the above description, steps S16 to S19 may be further divided into additional steps or combined into fewer steps, according to the implementation example of the present invention. Also, some steps may be omitted if necessary, and an order between operations may be changed.

The method of displaying an ultrasound image based on mixed reality according to the exemplary embodiment of the present invention may be implemented in the form of program commands that may be executed through various computer means and recorded on a computer-readable medium. The computer-readable recording medium may include a program command, a data file, a data structure or the like, alone or a combination thereof. The program commands recorded in the computer-readable recording medium may be specially designed and configured for the present invention or be known to those skilled in a field of computer software to be used. Examples of the computer-readable recording medium may include a magnetic medium such as a hard disk, a floppy disk, or a magnetic tape; an optical medium such as a compact disk read only memory (CD-ROM) or a digital versatile disk (DVD); a magneto-optical medium such as a floptical disk; and a hardware device specially configured to store and execute program commands, such as a ROM, a random access memory (RAM), a flash memory, or the like. Examples of the program commands include a high-level language code capable of being executed by a computer using an interpreter, or the like, as well as a machine language code made by a compiler. The above-described hardware device may be constituted to be operated as one or more software modules to perform an operation according to the present invention, and vice versa.

In addition, the above-described method of displaying an ultrasound image based on mixed reality may also be implemented in the form of a computer program or application that is stored in a recording medium and executed by a computer.

The above description of the present invention is for illustrative purposes, and those skilled in the art to which the present invention pertains will understand that it is possible to be easily modified to other specific forms without changing the technical spirit or essential features of the present invention. Therefore, it should be understood that the above-mentioned exemplary embodiments are exemplary in all aspects but are not limited thereto. For example, each component described as a single type may be implemented in a distributed manner, and similarly, components described as distributed may be implemented in a combined form.

It is to be understood that the scope of the present invention will be defined by the claims rather than the above-mentioned description and all modifications and alternations derived from the claims and their equivalents are included in the scope of the present invention.

1. A method of displaying an ultrasound image based on mixed reality, comprising:
obtaining the ultrasound image captured through an ultrasound device;
obtaining localization information of a probe of the ultrasound device;
converting the ultrasound image into a composite image to be output based on the mixed reality; and
outputting the composite image by overlaying the composite image on a reference image captured to include the probe.

2. The method of claim 1, wherein, in the outputting, the composite image is overlaid to be adjacent to an end area of the probe that appears in the reference image.

3. The method of claim 1, wherein, in the outputting, the composite image is overlaid on the reference image displayed on a screen of a head-mounted display worn by a user.

4. The method of claim 3, further comprising:
obtaining position information and field of view information of the head-mounted display; and
correcting a overlaid position of the composite image based on the position information and the field of view information.

5. The method of claim 1, further comprising:
receiving user input for capturing the composite image; and
displaying a fixed image in which the composite image is continuously maintained at a predetermined fixed position on the reference image based on the user input.

6. The method of claim 5, wherein, in the displaying of the fixed image, the fixed image is output by applying a predetermined transparency to the composite image.

7. The method of claim 5, further comprising:
extracting three-dimensional (3D) boundary information on a captured object of the ultrasound device based on color information of each of a plurality of fixed images in a state where the fixed image is individually displayed at a plurality of different fixed positions based on the user input applied multiple times; and
updating the composite image to include a virtual 3D image corresponding to the captured object based on the 3D boundary information.

8. The method of claim 1, further comprising:
receiving user input for adjusting a position of the composite image; and
adjusting a overlaid position of the composite image on the reference image based on the user input.

9. A method of displaying an ultrasound image based on mixed reality, comprising:
obtaining the ultrasound image captured through an ultrasound device;
obtaining localization information of a probe of the ultrasound device;
converting the ultrasound image into a composite image to be output based on the mixed reality in consideration of the localization information; and
transmitting the composite image to a user terminal.

10. The method of claim 9, wherein the user terminal includes a head-mounted display worn by a user operating the ultrasound device.

11. The method of claim 9, further comprising:
receiving user input for capturing the composite image; and
transmitting, to the user terminal, a fixed image in which the composite image is continuously maintained at a predetermined fixed position on a reference image captured to include the probe based on the user input.

12. The method of claim 11, further comprising:
extracting three-dimensional boundary information on a captured object of the ultrasound device based on color information of each of a plurality of fixed images in a state where the fixed image is individually displayed at a plurality of different fixed positions based on the user input applied multiple times; and
transmitting, to the user terminal, the composite image updated to include a virtual 3D image corresponding to the captured object based on the 3D boundary information.

13. An apparatus for providing ultrasound image display service based on mixed reality, comprising:
a receiving unit that obtains an ultrasound image captured through an ultrasound device and obtains localization information of a probe of the ultrasound device;
a processing unit that converts the ultrasound image into a composite image to be output based on the mixed reality in consideration of the localization information; and
a transmitting unit that transmits the composite image to a user terminal.

14. A system for displaying an ultrasound image based on mixed reality, comprising:
an ultrasound device that captures an ultrasound image using a probe;
a service providing device that obtains the ultrasound image and localization information of the probe, and converts the ultrasound image into a composite image to be output based on the mixed reality; and
a user terminal that receives the composite image from the service providing device and outputs the composite image by overlaying the composite image on a reference image captured to include the probe.

15. The system of claim 14, further comprising:
a tracking device that is disposed relative to the probe, measures the localization information, and transmits the measured localization information to the service providing device.
